(19) United States
(12) Patent Application Publication
(10) Pub. No.: US 2026/0259194 A1
(43) Pub. Date: Sep. 3, 2026

(54) METHOD OF MONITORING MESENCHIMAL STEM CELL (MSC) POTENCY AND IMPROVING MSC DIFFERENTIATION POTENTIAL

(71) Applicant: AGENCY FOR SCIENCE, TECHNOLOGY AND RESEARCH, Singapore (SG)

(72) Inventors: Tongming LIU, Singapore (SG); Cool SIMON, Singapore (SG)

(21) Appl. No.: 18/995,728

(22) PCT Filed: Jun. 28, 2023
(86) PCT No.: PCT/SG2023/050457

§ 371 Date: Jan. 16, 2025

(30) Foreign Application Priority Data
Sep. 14, 2022 (SG) 10202250995W

(51) Int. Cl. G01N 33/50; A61K 35/28; C12N 5/0775; C12N 15/86; G01N 33/58

(52) U.S. Cl. CPC G01N 33/5005; A61K 35/28; C12N 5/0668; C12N 15/86; G01N 33/582; C12N 2510/00; C12N 2740/15043; G01N 2333/4703

(57) ABSTRACT

Disclosed herein are a method of identifying therapeutically potent mesenchymal stem cells (MSCs) and a method of distinguishing MSCs from human fibroblasts, comprising detecting HOXA9 expression in the cell. Also disclosed is a method of treating diseases in a subject in need of MSCs, comprising modifying MSCs to overexpress HOXA9 and administering the modified MSCs to the subject in need.

### REFERENCE TO RELATED APPLICATIONS

This application claims the priority to Singapore patent application Ser. No. 10202250995W, filed 14 Sep. 2022, the contents of which are incorporated herein by reference.

### TECHNICAL FIELD

The present disclosure relates generally to distinguishing mesenchymal stem cells (MSCs), preferably distinguishing, making and/or using MSCs that are more potent.

### BACKGROUND

The following discussion of the background to the invention is intended to facilitate an understanding of the present invention only. It should be appreciated that the discussion is not an acknowledgement or admission that any of the material referred to was published, known or part of the common general knowledge of the person skilled in the art in any jurisdiction as at the priority date of the invention.

Mesenchymal stem cells (MSCs) represent the most clinically used stem cells in >1000 registered clinical trials with efficacy against over 30 human diseases. Despite these advances, there is a dearth of biomarkers which distinguish between cells with high therapeutic potency and those with low potency. Although human mesenchymal stem cells (MSCs) represent the most clinically used stem cells for regenerative medicine, little is known about biomarkers associated with MSC stemness/potency (or quality), less is known about the molecular basis of MSC stemness/potency.

It is difficult to ensure that a particular batch of MSCs will be able to be effective in treatment or differentiation because there is no way to evaluate the quality of MSCs during biomanufacturing for large-scale manufacturing.

HOXA9 is a transcription factor homeobox genes. It is reported to be involved in embryonic development, morphogenesis, dysregulation of red blood cells in leukaemia, and to play a role in blood cell lineages.

There exists a need to classify, identify distinguish and/or make different classes of MSCs and alleviate at least one of the aforementioned problems.

### SUMMARY

Methods of classifying, identifying distinguishing and making different classes of MSCs and potent MSCs are envisaged.

Accordingly, an aspect of the invention refers to a method of identifying therapeutically potent Mesenchymal stem cells (MSCs) comprising detecting HOXA9 expression; wherein an expression value at or over a first predetermined value indicates the MSCs are therapeutically potent.

According to another aspect of the invention refers to a method of distinguishing Mesenchymal stem cells (MSCs) from human fibroblasts comprising detecting HOXA9 expression in the cell; wherein an expression value at or over a first predetermined value indicates the cell is an MSCs and an expression value below the second predetermined value indicates the cell is a human fibroblast.

According to another aspect of the invention refers to potent mesenchymal stem cells (MSCs) comprising stem cells modified to overexpress HOXA9, wherein the modified cells demonstrate adipogenenic, osteogenic and chondrogenic differentiation potential.

According to another aspect of the invention refers to potent mesenchymal stem cells as described herein above for use in treating a disease

According to another aspect of the invention refers to a method of treating diseases in a subject in need of Mesenchymal stem cells (MSCs) comprising modifying Mesenchymal stem cells (MSCs) to overexpress HOXA9 and administering the modified MSCs to the subject in need.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of non-limiting examples only, embodiments of the present invention,

FIG. 1: (A) step-wise iPSC-MSC platform which recapitulated developmental stages of MSCs, gene expression profiles during MSC development were compared using microarray and a gene list specifically expressed in both iPS-MSCs and bone marrow-MSCs, but not expressed in other stage of cells such as human fibroblasts, primitive streak and mesoderm was generated. (B) Microarray data showed that HOXA9 is one of the top transcription factors specifically expressed in both iPS-MSCs and bone marrow-MSCs, QPCR data were consistent with microarray data

FIG. 2: HOXA9 knockdown abolished the proliferation of MSCs (A) To determine the role of HOXA9 in the MSCs, MSCs were infected by lentiviral shRNA against HOXA9. QPCR and immunofluorescence showed HOXA9 was knocked-down (B) the effects of HOXA9 knockdown on the proliferation of MSCs, compared to scrambled MSCs demonstrated that scrambled MSCs proliferated well whereas knockdown MSCs lose ability to proliferate.

FIG. 3: HOXA9 knockdown greatly decreased the CFU-F an important property of MSCs. Compared with scrambled control, HOXA9 knockdown greatly decreased CFU-F of MSCs.

FIG. 4: FACS data showed that HOXA9 knockdown decreased the percentage of CD73+, CD90+ and CD105+MSCs by at least 10% compared with scrambled control.

FIG. 5: (A) showing that HOXA9 knockdown promoted the senescence of MSCs, (B) HOXA9 knockdown decreased the telomere length of MSCs.

FIG. 6: (A) Compared with scrambled control, HOXA9 knockdown decreased oil red stain for oil droplet in adipogenesis, AP stain for alkaline phosphatase in osteogenesis and alcian blue stain for proteoglycan in chondrogenesis (B) Stain data were validated by qPCR for lineage specific genes, compared with scrambled control, HOXA9 knockdown decreased adipogenenic genes (CEBPα, PPARγ, LPL and αP2), osteogenic genes (OC, OPN and ALP) and chondrogenic genes (COL2A1, AGC1, SOX9 and COL10A1) and HOXA9 knockdown decreased alkaline phosphatase (AP) activity.

FIG. 7: Effects of HOXA9 specific inhibitor DB818 on MSCs, (A) Compared with DMSO control, HOXA9 inhibitor also abolished MSC proliferation (B) HOXA9 inhibitor abolished CFU-F. (C) HOXA9 inhibitor decreased oil red stain for oil droplet in adipogenesis, AP stain for alkaline phosphatase in osteogenesis and alcian blue stain for proteoglycan in chondrogenesis and (D) HOXA9 inhibitor decreased adipogenenic genes (CEBPα, PPARγ, LPL and αP2), osteogenic genes (RUNX2 and COL1A1) and chondrogenic genes (COL2A1, AGC1, SOX9 and COL10A1).

FIG. 8: HOXA9 overexpression improved the differentiation potential of MSCs, (A) HOXA9 was overexpressed with lentiviral system. (B) Compared with empty control, HOXA9 overexpression improved oil red stain for oil droplet in adipogenesis, AP stain for alkaline phosphatase in osteogenesis and alcian blue stain for proteoglycan in chondrogenesis. (C) Stain data were validated by qPCR for lineage specific gene which increased adipogenenic genes (CEBPα, PPARγ, LPL and αP2), osteogenic genes (OC, OPN and ALP) and chondrogenic genes (COL2A1, AGC1, SOX9 and COL10A1).

FIG. 9: (A) HOXA9 expression correlated with MSC important genes such as RUNX2, SOX9, TWIST1, TWIST2, EZH2 and p16INKA4A (B) Compared with scrambled control, HOXA9 knockdown decreased MSC important genes, including TWIST1, TWIST2, EZH2 as well as secreting genes IFNγ, IDO1, IL6, IL8, (C) HOXA9 knockdown decreased the expression of MSC important proteins such as RUNX2, SOX9, TWIST1, TWIST2, EZH2 (D) HOXA9 overexpression increased the expression of TWIST2 and EZH2 (E) ChIP-seq data showed that HOXA9 most bound to the promoter and distal intergenic regions of target genes, (F) the direct target genes of HOXA9 were associated with cellular senescence, cell cycle, FOXO signaling pathway and hippo signaling pathway.

FIG. 10: The immortalized MSCs (UE7T-13) were infected with lentiviral HOXA9 reporter consisting of HOXA9 promoter driven eGFP, (A) the strongest and the weakest eGFP cells were sorted based on fluorescence expression. (B) The strong reporter MSCs express higher EZH2, MKI67, IDO1, IFNγ and IL1R1 than weak reporter MSCs. (C) Compared with non-infected MSCs and weak reporter MSCs, strong reporter MSCs have longer telomere length. (D) Upon osteogenesis for 2 weeks, strong reporter MSCs accumulated more calcium deposit shown by alizarin red stain and express higher osteogenic genes such as OC, OPN and ALP than weak reporter MSCs.

FIG. 11: (A) HOXA9 knockdown changed gene expression profile of MSCs. (B) Compared with control, 1345 genes were upregulated whereas 1682 genes were downregulated in knockdown MSCs. (C) GO analysis showed that HOXA9 knockdown affected genes associated with transforming growth factor beta binding, growth factor binding, extracellular matrix structural constituent, and positive regulation of cell motility and migration. (D) KEGG analysis showed that HOXA9 is associated with MAPK, Rap1, CGMP-PKG, Apelin and AGE-RAGE signaling pathways.

FIG. 12: To understand how HOXA9 regulates MSC stemness, HOXA9 binding sites at early and late passage of MSCs are determined by chromatin immunoprecipitation assays with sequencing (ChIP-seq). (A) HOXA9 most binds to the promoters. (B) HOXA9 binding sites associate with H3K4me3 binding sites for active genes, but are depleted in H3K4me27 binding sites for inactive genes. (C) Most importantly, HOXA9 binding sites decreases more at late passage of MSCs compared with early passage of MSCs, this maybe associates with the loss of MSC stemness at late passage. (D) Total binding sites decreased from 8288 at early passage of MSCs to 3136 at late passage of MSCs, active binding sites decreased from 3985 at early passage of MSCs to 1914 at late passage of MSCs. (E) HOXA9 binding sites overlapped with FOS, STAT4, SMAD4, MEIS1 and TWIST2 binding sites. (F) HOXA9 binding sites are also associated with mitotic cell cycle, cell proliferation, cartilage development, skeletal system morphogenesis and chondrocytes, suggest that HOXA9 regulates MSC stemness by binding to self-renewal and differentiation gene of MSCs.

FIG. 13: (A) ChIP-seq data showed that HOXA9 directly binds to TWIST1, the important MSC gene associated with MSC self-renewal and differentiation. (B) Rescue experiment showed that HOXA9 knockdown greatly decreased MSC proliferation whereas TWIST1 overexpression increased MSC proliferation. Most importantly, TWIST1 overexpression can rescue the effects of HOXA9 knockdown on MSC proliferation. (C) Based on ChIP-seq, HOXA9 knockdown, inhibitor and overexpression data, the mechanism of HOXA9 regulating MSC stemness is proposed, HOXA9 directly binds to TWIST1, which Induces EZH2 recruitment regulating histone methylation to regulate MSC self-renewal and differentiation potential.

FIG. 14: To test if HOXA9 overexpression improves bone formation in vivo, HOXA9 overexpressing and empty control MSCs were seeded onto the bone scaffold of PCL-TCP with help of fibrin glue, after 1 week of pre-differentiation in osteogenic medium, differentiated cells were subcutaneously transplanted into immunodeficient mice for 8 weeks. μCT data showed that HOXA9 overexpression improved bone formation in vivo compared with empty control.

### DETAILED DESCRIPTION

Throughout this document, unless otherwise indicated to the contrary, the terms “comprising”, “consisting of”, “having” and the like, are to be construed as non-exhaustive, or in other words, as meaning “including, but not limited to”.

Furthermore, throughout the document, unless the context requires otherwise, the word “include” or variations such as “includes” or “including” will be understood to imply the inclusion of a stated integer or group of integers but not the exclusion of any other integer or group of integers.

Throughout the description, it is to be appreciated that the term ‘HOXA9 gene expression’ and its plural form include a protein expression product initiated by a HOXA9 gene promotor nucleic acid. In various embodiments the protein expression product initiated by a HOXA9 gene promotor nucleic acid comprises a homeobox protein Hox-A9 which is the expression product of the HOXA9 gene found at GenBank accession number NG_029923 and listed here as SEQ ID NO:1. In various embodiments the protein expression product initiated by a HOXA9 gene promotor nucleic acid comprises an enhanced green fluorescence protein (eGFP) or a similar fluorescence tag wherein the HOXA9 gene promotor nucleic acid is linked upstream of a nucleic acid expressing an eGFP. In various embodiments the HOXA9 gene promotor nucleic acid comprises at least 90%, or 91%, or 92%, or 93%, or 94%, or 95%, or 96%, or 97%, or 98%, or 99%, or 100% sequence identity with the following nucleic acid sequence. SEQ ID NO:2 gccccgggacaggccacatcggtgcgggcctcccaggttccggagctgcggggtctcttaggcgaggctgccttttcccaaac cgaacttgccttccattcatgccacttgtagttttttccccagctgggattcacggagcgcaaccaggcttgcagcgctcatggtta gagcctctgaggctggagcacagggctgggtcgccagccgcctgcgcctgggaatcctgattgccagctgatgagaaaggc gggctgggcgcgcgtgtgcgtggggtcgagggccggggaccgagcgcgccgcacaaccaaccaggccctcaaaaccttc gccctggtggcggctggccgctccctcctggccagctcctccgtggggtcctcgtagcaaaggcgaatttaagggttgcccgg gcgcccctcgctccagggggtagctgtggggacctacacccgcggtactccctgagcggccggtccctgcctggagtgccc tggtagggccggcggcggctccgtttgggacggatcctgcgttgaatttgacttttcgagggcggccgcgggtaaactcgcctct cccggggaccgcagggattatttacagggagctcgccaaccaaacacaacagtctaacctttccaagtcctcgtaaatttttac agctgggagccacggcgaggcaaacgaatctgttggtcgtttccgacttcccgccagcctgtgtggcttctgaaacaataactc cttatgaaatatcataaatatagatttaaatacagtagagcgacaatgcgatttggctgcttttttatggcttcaattattgtctaatttta tgtgaggggctccgctggccgcactcgcacgcgggacccgcgccttcttgatggcgtgattaattgtgatataaaatagtccgct taagaagtgtgtgtatggggggggagacgggagagtacagagacaaggctagatttgatcttttaatcgtcgttggccacaatt aaaacaaaccccatcgtagagcggcacgatccctttacataaaaacatatggcttttgctataaaaattatgactgcaaaacat cggaccattaatagcgtgcggagtgatttacgcgttattgttctgctggacgggcacgtgacgcgcacggccaatgggggcgc gggcgccggcaacttattaggtgactgtacttcccccccggtgccaccaagttgttacatgaaatctgcagtttcataatttccgtg ggtcgggccgggc. In various embodiments the nucleic acid expressing eGFP comprises at least 98% sequence identity with the following nucleic acid sequence SEQ ID NO:3 atggtgagcaagggcgaggagctgttcaccggggtggtgcccatcctggtcgagctggacggcgacgtaaacggccacaa gttcagcgtgtccggcgagggcgagggcgatgccacctacggcaagctgaccctgaagttcatctgcaccaccggcaagct gcccgtgccctggcccaccctcgtgaccaccctgacctacggcgtgcagtgcttcagccgctaccccgaccacatgaagcag cacgacttcttcaagtccgccatgcccgaaggctacgtccaggagcgcaccatcttcttcaaggacgacggcaactacaaga cccgcgccgaggtgaagttcgagggcgacaccctggtgaaccgcatcgagctgaagggcatcgacttcaaggaggacgg caacatcctggggcacaagctggagtacaactacaacagccacaacgtctatatcatggccgacaagcagaagaacggc atcaaggtgaacttcaagatccgccacaacatcgaggacggcagcgtgcagctcgccgaccactaccagcagaacacccc catcggcgacggccccgtgctgctgcccgacaaccactacctgagcacccagtccgccctgagcaaagaccccaacgaga agcgcgatcacatggtcctgctggagttcgtgaccgccgccgggatcactctcggcatggacgagctgtacaagtaa. In various embodiments expression of the HOXA9 gene is listed as uniprot Number P31269. In various embodiments expression of the HOXA9 gene comprises at least 98% sequence identity with the following amino acid sequence SEQ ID NO:4 MATTGALGNYYVDSFLLGADAADELSVGRYAPGTLGQPPRQAATLAEHPDFSPCSFQSK ATVFGASRNPVHAAGANAVPAAVYHHHHHHPYVHPQAPVAAAAPDGRYMRSWLEPTPG ALSFAGLPSSRPYGIKPEPLSARRGDCPTLDTHTLSLTDYACGSPPVDREKQPSEGAFSE NNAENESGGDKPPIDPNNPAANWLHARSTRKKRCPYTKHQTLELEKEFLFNMYLTRDRR YEVARLLNLTERQVKIWFQNRRMKMKKINKDRAKDE.

Unless defined otherwise, all other technical and scientific terms used herein have the same meaning as is commonly understood by a skilled person to which the subject matter herein belongs.

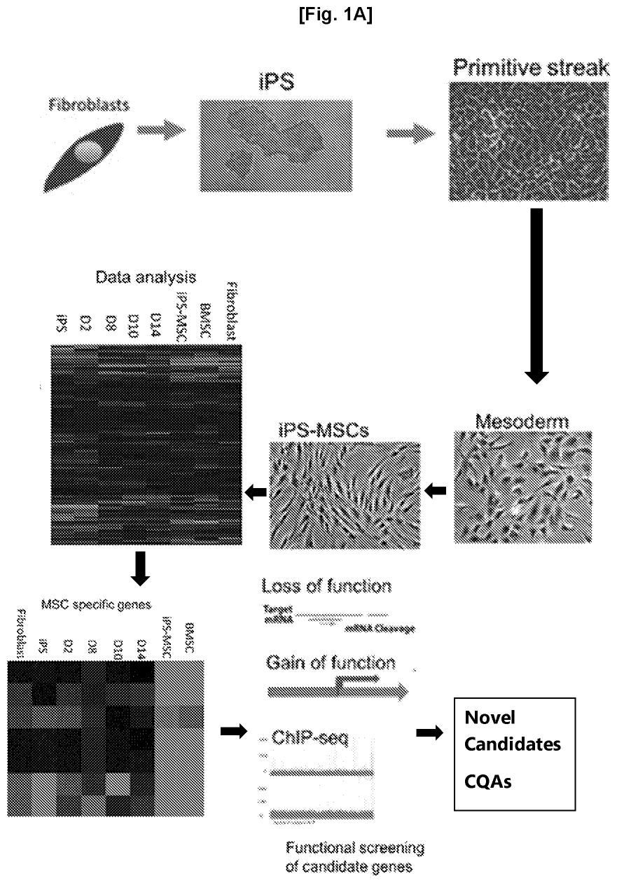

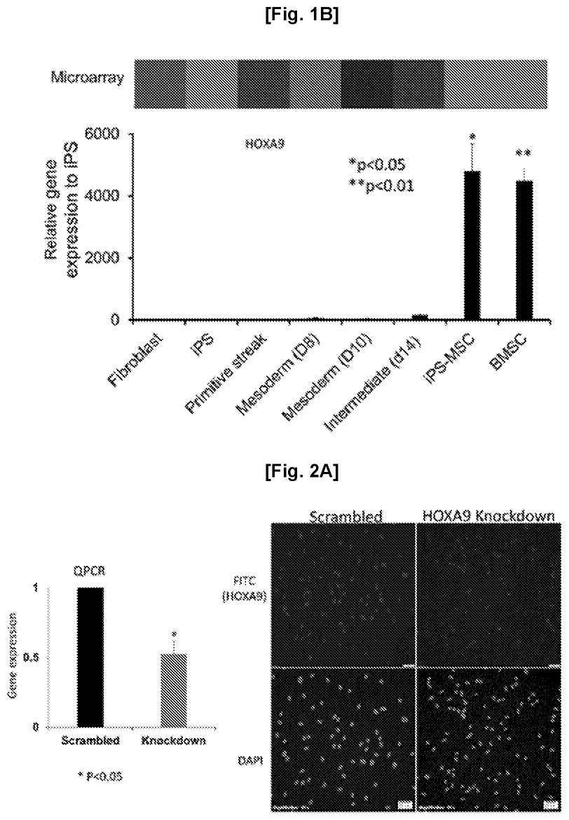

So far, little is known about biomarkers associated with MSC stemness/potency, less is known about molecular basis of MSC stemness/potency. Using a unique and step-wise platform technology developed to generate MSCs from iPSCs (iPSC-MSCs) by a series of functional genomic screening assays (see [FIG. 1A]), HOXA9 was identified to be critical to MSC stemness/potency, which was much downregulated in late-passaged MSCs. Knockdown of HOXA9 reduced expression of mRNA transcripts and proteins linked to MSC function and abolished proliferation and colony formation (CFU-F). Moreover, downregulation or inhibition of HOXA9 expression accelerated MSC senescence and a decrease in the expression of cell surface antigens linked to the MSC phenotype was observed, multi-linage differentiation was greatly impaired. Notably, overexpression resulted in improved multi-lineage differentiation. This study develops a biomarker that can be used to monitor the quality of MSCs or evaluate MSC media during biomanufacturing for large-scale manufacturing. The identified HOXA9 also represents a novel and promising strategy for gene therapy in regenerative medicine.

To identify novel biomarker for evaluation of MSC quality and understanding of MSC stemness/potency, gene list specifically expressed in MSCs were generated using a unique and step-wise iPSC-MSC platform listed in [FIG. 1A]. HOXA9 was one of top transcription factors specifically expressed in both iPSC-MSCs and bone marrow-MSCs. Gene functionality of HOXA9 was studied by loss or gain of function and using a specific inhibitor, showing that HOXA9 is a key transcription factor to MSC self-renewal and differentiation.

HOXA9 expression is a key transcription factor to MSC stemness/potency, therefore HOXA9 reporter can be used to monitor MSC stemness/potency (or quality) or evaluate the different MSC media and/or identify different subpopulations of MSC cells.

Genetically modified MSCs with HOXA9 showed improved differentiation potential, therefore HOXA9 represents one promising gene therapy strategy for regenerative medicine.

HOXA9 was shown to be specifically expressed in iPSC-MSCs and bone marrow-MSCs. So far, little is known about HOXA9 in the role of MSCs. Here for the first time HOXA9 is shown to be a key transcription factor to MSC stemness/potency by various means including HOXA9 knockdown, inhibition of HOXA9, overexpression of HOXA9, RNA-seq and ChIP-seq. This novel finding develops a biomarker that can be used in various embodiments to monitor the quality of MSCs or evaluate MSC media during biomanufacturing for large-scale manufacturing. In various embodiments the identified HOXA9 also represents a novel and promising strategy for gene therapy in regenerative medicine.

In various embodiments HOXA9 expression or expression of HOXA9 reporter may be used to monitor MSC stemness/potency (or quality) or evaluate MSC media.

This has the advantage of allowing identification or selection of sub-populations of MSCs that have better multipotency capable of differentiating into three different cell types including adipogenic, osteogenic and chondrogenic cells.

According to various embodiments there is a method of identifying therapeutically potent Mesenchymal stem cells (MSCs) comprising detecting HOXA9 expression; wherein an expression value at or over a first predetermined value indicates the MSCs are therapeutically potent.

In various embodiments the method of identifying therapeutically potent Mesenchymal stem cells (MSCs) comprises detecting HOXA9 expression; wherein an expression value at or over a first predetermined value indicates the MSCs are therapeutically potent and a value below the first predetermined value indicates the cells are not as potent MSCs or are human fibroblasts. Allowing MSCs to be separated into different sub-populations.

As used herein a therapeutically potent Mesenchymal stem cells (MSCs) comprises a mesenchymal stem cell multipotent cells capable of differentiating into three different cell types including adipogenenic, osteogenic and chondrogenic cells. Similarly, MSCs that are not as potent MSCs will not be multipotent and may not be capable of differentiating into three different cell types. For example, in various embodiments the not as potent MSCs may only be capable of differentiating into one or two different cell types. In various embodiments the not as potent MSCs may only be capable of differentiating into adipogenenic cell types and not capable of differentiating into osteogenic and chondrogenic cells. In various embodiments the not as potent MSCs may only be capable of differentiating into osteogenic cell types and not capable of differentiating into adipogenenic and chondrogenic cells. In various embodiments the not as pluripotent MSCs may only be capable of differentiating into chondrogenic cell types and not capable of differentiating into adipogenenic and osteogenic cells.

In various embodiments HOXA9 expression in the cell is determined by any known method of measuring protein or RNA expression including but not limited to western blotting, ELISA assays, qPCR, or any other method of measuring protein or RNA expression known to a person skilled in the art. In various embodiments HOXA9 expression in the cell may be determined from downstream results such as ability to proliferate, CFU-F property, MSC surface antigen profile, of CD73+, CD90+ and CD105+, whether cells are positive for senescence indication using β-gal stain or telomere length, and/or the differentiation potential of MSCs into three lineages.

In various embodiments the first predetermined value may about 1.3 to 2 times more than a knockdown or inhibition or exhaustion of HOXA9 expression. In various embodiments the first predetermined value may about 1.4 to 2 times more than a knockdown or inhibition or exhaustion of HOXA9 expression. In various embodiments the first predetermined value may about 1.5 to 2 times more than a knockdown or inhibition or exhaustion of HOXA9 expression. In various embodiments the first predetermined value may about 1.6 to 2 times more than a knockdown or inhibition or exhaustion of HOXA9 expression. In various embodiments the first predetermined value may about 1.7 to 2 times more than a knockdown or inhibition or exhaustion of HOXA9 expression. In various embodiments the first predetermined value may about 1.8 to 2 times more than a knockdown or inhibition or exhaustion of HOXA9 expression. In various embodiments the first predetermined value may about 1.9 to 2 times more than a knockdown or inhibition or exhaustion of HOXA9 expression.

In various embodiments the first predetermined value may about 1.3 to 2 times more than a knockdown of HOXA9 expression. In various embodiments the first predetermined value may be determined by dividing a sample from a batch of MSCs into two portions and introducing a different shRNA, into each portion wherein a shRNA that knocks-down HOXA9 expression is introduced into the first portion and a scrambled shRNA is introduced into the second portion wherein when the expression of HOXA9 in the second portion is from 1.3 to 2 times more than the expression of HOXA9 in the first portion the batch of MSCs is evaluated to have HOXA9 expression value at or over a first predetermined value indicating that the MSCs in that batch are therapeutically potent. In various embodiments the shRNA that HOXA9 knocks-down is SEQ ID NO:5 expression AGCCGGCCTTATGGCATTAAAC. In various embodiments the shRNA that knocks-down HOXA9 expression is SEQ ID NO:6 TTCTCCTCCAGTTGATAGAGAA. In various embodiments the scrambled shRNA nucleic acid sequence used is SEQ ID NO:7 CCTAAGGTTAAGTOGCCCTCG.

In various embodiments the first predetermined value may about 1.3 to 2 times more than inhibition of HOXA9 expression. In various embodiments the first predetermined value may be determined by dividing a sample from a batch of MSCs into two portions and introducing a HOXA9 inhibitor DB818 into a first portion and introducing DMSO into a second portion wherein when the expression of HOXA9 in the second portion is from 1.3 to 2 times more than the expression of HOXA9 in the first portion, the batch of MSCs is evaluated to have HOXA9 expression value at or over a first predetermined value indicating that the MSCs in that batch are therapeutically potent.

In various embodiments the first predetermined value may about 1.3 to 2 times more than exhaustion of HOXA9 expression. In various embodiments the first predetermined value may be determined by taking a first sample at early passage stage and second sample at late passage stage and comparing HOXA9 gene expression in both the samples, wherein when the expression of HOXA9 in the first sample is from 1.3 to 2 times more than the expression of HOXA9 in the second sample the batch of MSCs is evaluated to have HOXA9 expression value at or over a first predetermined value indicating that the MSCs in that batch are therapeutically potent.

According to various embodiments there is a method of distinguishing Mesenchymal stem cells (MSCs) from human fibroblasts comprising detecting HOXA9 expression in the cell; wherein an expression value at or over a second predetermined value indicates the cell is an MSCs and an expression value below the second predetermined value indicates the cell is a human fibroblast.

Both human MSCs and human fibroblasts are similar. HOXA9 can be used to distinguish human MSCs from human fibroblasts. The former, human MSCs, highly express HOXA9 whereas the latter, human fibroblasts, express very low or no HOXA9.

In various embodiments the second predetermined value may about 0.5 to 2 times, 0.6 to 2 times, 0.7 to 2 times, 0.8 to 2 times, 0.9 to 2 times, 1 to 2 times, 1.1 to 2 times, 1.2 to 2 times, 1.3 to 2 times, 1.4 to 2 times, 1.5 to 2 times, 1.6 to 2 times, 1.7 to 2 times, 1.8 to 2 times, 1.9 to 2 times more than a knockdown or inhibition or exhaustion of HOXA9 expression in a corresponding cell sample or batch. In various embodiments the second predetermined value may be determined by any of the methods described herein above to determine the first predetermined value. In various embodiments the first and second predetermined value may be the same or identical where the MSCs are therapeutically potent. As would be understood by a person skilled in the art where no or very little HOXA9 is expressed in human fibroblasts then knockdown or inhibition or exhaustion of HOXA9 expression in the human fibroblasts would vary very little from the human fibroblasts where there is no knockdown or inhibition or exhaustion. This allows accurate classification of different cell types for example human fibroblasts, not potent MSCs and potent MSCs.

In various embodiments the method further comprises introducing a HOXA9 reporter into the cells wherein the HOXA9 reporter comprises a fluorescence tag such as an enhanced green fluorescence protein (eGFP) driven by a HOXA9 promoter; incubating the cells; and measuring the fluorescence of the HOXA9 reporter to determine the equivalent of detecting HOXA9 expression at or over the first predetermined value or HOXA9 expression at or over the second predetermined level.

In various embodiments the MSC's may be sorted into MSCs expressing the HOXA9 reporter at or over the first predetermined value or the HOXA9 reporter at or over the second predetermined value. In various embodiment the MSC's are sorted using cell sorting technology such as fluorescence activated cell sorting (FACS) or immunomagnetic cell sorting. In various embodiments where the MSCs are modified to overexpress HOXA9 reporter to facilitate FACS.

In various embodiments the therapeutically potent MSCs express genes selected from any one of EZH2, MIK67, IDO1 IFNγ and a combination thereof at or above expression of these genes in human fibroblasts or in MSC not as potent MSCs that are not multipotent.

In various embodiments the MSCs expressing HOXA9 at or over the first predetermined value expressed at least one gene selected from the group consisting of RUNX2, SOX9, TWIST1, TWIST2, EZH2 and p16INKA4A at an increased amount compared to the expression of at least one the genes in MSCs expressing HOXA9 below the first predetermined value.

As used herein, the expression of the RUNX2 gene is Runt-related transcription factor 2 (RUNX2) or also known as core-binding factor subunit alpha-1 (CBF-alpha-1). In various embodiments expression of the RUNX2 gene is listed as uniprot Number Q13950 and listed here as SEQ ID NO:8.

As used herein, the expression of the SOX9 gene Transcription factor SOX-9. In various embodiments expression of the SOX9 gene is listed as uniprot Number P48436 and listed here as SEQ ID NO:9.

As used herein, the expression of the TWIST1 gene is Twist-related protein 1 (TWIST1) also known as class A basic helix-loop-helix protein 38 (bHLHa38). In various embodiments expression of the TWIST1 gene is listed as uniprot Number Q15672 and listed here as SEQ ID NO:10. Without being bound to any theory the inventors postulate that HOXA9 binds directly to TWIST1 which induces EZH2 recruitment, regulating histone methylation resulting in regulation of MSCs self-renewal and differentiation potential as depicted in [FIG. 13C].

As used herein, the expression of the TWIST2 gene is Twist-related protein 2 (TWIST2). In various embodiments expression of the TWIST2 gene is listed as uniprot Number Q8WVJ9 and listed here as SEQ ID NO:11.

As used herein, the expression of the EZH2 gene is enhancer of zeste homolog 2 or Histone-lysine N-methyltransferase. In various embodiments expression of the EZH2 gene is listed as uniprot Number Q15910 and listed here as SEQ ID NO:12.

As used herein, the expression of the p16INKA4A gene is p16, also known as p16INK4a, cyclin-dependent kinase inhibitor 2A, CDKN2A, multiple tumor suppressor 1 and numerous other synonyms). In various embodiments expression of the p16INKA4A gene is listed as uniprot Number P42771 or Q8N726 and listed here as SEQ ID NOS: 13 (Q8N726) and 14 (P42771 isoform).

In various embodiments the MSCs expressing HOXA9 at or over the predetermined value also expressed at least two genes selected from the group consisting of RUNX2, SOX9, TWIST1, TWIST2, EZH2 and p16INKA4A at an increased amount compared to the expression of at least two of the same genes in MSCs expressing HOXA9 below the predetermined value. In various embodiments the at least two genes comprise or consist if TWIST1 and EZH2. In various embodiments the MSCs expressing HOXA9 at or over the predetermined value also expressed at least three genes selected from the group consisting of RUNX2, SOX9, TWIST1, TWIST2, EZH2 and p16INKA4A at an increased amount compared to the expression of at least three of the same genes in MSCs expressing HOXA9 below the predetermined value. In various embodiments the MSCs expressing HOXA9 at or over the predetermined value also expressed at least four genes selected from the group consisting of RUNX2, SOX9, TWIST1, TWIST2, EZH2 and p16INKA4A at an increased amount compared to the expression of at least four of the same genes in MSCs expressing HOXA9 below the predetermined value. In various embodiments the MSCs expressing HOXA9 at or over the predetermined value also expressed at least five genes selected from the group consisting of RUNX2, SOX9, TWIST1, TWIST2, EZH2 and p16INKA4A at an increased amount compared to the expression of at least five the same genes in MSCs expressing HOXA9 below the predetermined value. In various embodiments the MSCs expressing HOXA9 at or over the predetermined value also expressed the genes of RUNX2, SOX9, TWIST1, TWIST2, EZH2 and p16INKA4A at an increased amount compared to the expression of the genes in MSCs expressing HOXA9 below the predetermined value.

According to various embodiments there is potent mesenchymal stem cells (MSCs) comprising stem cells modified to overexpress HOXA9, wherein the modified cells demonstrate adipogenenic, osteogenic and chondrogenic differentiation potential.

In various embodiments therapeutically potent Mesenchymal stem cells (MSCs) comprises a mesenchymal stem cell capable of differentiating into three different cell types including adipogenenic, osteogenic and chondrogenic cells. In various embodiments the therapeutically potent MSCs are multipotent.

In various embodiments the modified MSCs are further modified to express a HOXA9 reporter comprising a fluorescence tag protein driven by a HOXA9 promoter. In various embodiments the fluorescence tag protein comprises an enhanced green fluorescence protein (eGFP).

In various embodiments expression of TWIST2 and EZH2 was increased compared to the expression of these genes in MSCs that are not modified to overexpress HOXA9.

In various embodiments the modified cells express at least one gene selected from the group consisting of CEBPα, PPARγ, LPL, αP2, OC, OPN, ALP, COL2A1, AGC1, SOX9 and COL10A1.

As used herein, the expression of the CEBPα gene is CCAAT enhancer-binding protein alpha a protein transcription factor. In various embodiments expression of the CEBPα gene is listed as uniprot Number P49715 and listed here as SEQ ID NO: 15.

As used herein, the expression of the PPARγ gene is Peroxisome proliferator-activated receptor gamma (PPAR-γ or PPARG), also known as the glitazone reverse insulin resistance receptor, or NR1C3 (nuclear receptor subfamily 1, group C, member 3) is a type II nuclear receptor. In various embodiments expression of the PPARγ gene is listed as uniprot Number P37231 and listed here as SEQ ID NO: 16.

As used herein, the expression of the LPL gene is Lipoprotein lipase (LPL) (EC 3.1.1.34, systematic name triacylglycerol acylhydrolase (lipoprotein-dependent). In various embodiments expression of the LPL gene is listed as uniprot Number P06858 and listed here as SEQ ID NO:17.

As used herein, the expression of the αP2 gene is Adipocyte Fatty Acid Binding Protein. In various embodiments expression of the αP2 gene is listed as uniprot Number P15090 and listed here as SEQ ID NO:18.

As used herein, the expression of the OC gene is osteocalcin. In various embodiments expression of the OC gene is listed as uniprot Number P02818 and listed here as SEQ ID NO:19.

As used herein, the expression of the OPN gene is osteopontin. In various embodiments expression of the OPN gene is listed as uniprot Number P10451 and listed here as SEQ ID NO:20.

As used herein, the expression of the ALP gene is alkaline phosphatase. In various embodiments expression of the ALP gene is listed as uniprot Number A0A024RAB4 and listed here as SEQ ID NO:21.

As used herein, the expression of the COL2A1 gene is Collagen Type II Alpha 1 Chain. In various embodiments expression of the COL2A1 gene is listed as uniprot Number P02458 and listed here as SEQ ID NO:22.

As used herein, the expression of the AGC1 gene is aggrecan. In various embodiments expression of the AGC1 gene is listed as uniprot Number P16112 and listed here as SEQ ID NO:23.

As used herein, the expression of the COL10A1 gene is Collagen Type X Alpha 1 Chain. In various embodiments expression of the COL10A1 gene is listed as uniprot Number Q03692 and listed here as SEQ ID NO:24.

In various embodiments expression of adipogenenic genes CEBPα, PPARγ, LPL and αP2 was higher than the expression of these genes in MSCs that are not modified to overexpress HOXA9.

In various embodiments expression of osteogenic genes OC, OPN and ALP was higher than the expression of these genes in MSCs that are not modified to overexpress HOXA9.

In various embodiments expression of chondrogenic genes COL2A1, AGC1, SOX9 and COL10A1 was higher than the expression of these genes in MSCs that are not modified to overexpress HOXA9.

According to various embodiments the potent mesenchymal stem cells discussed herein above are suitable for use in treating a disease.

In various embodiments the cells are suitable for use in treating bone and/or cartilage repair. In various embodiments the disease comprises bone and/or cartilage repair. In various embodiments the disease comprises a disease requiring regenerative repair of adipogenenic tissue, osteogenic tissue or chondrogenic tissue.

According to various embodiments the use of potent mesenchymal stem cells discussed herein above in the manufacture of a medicament for treating regenerative repair of adipogenenic tissue, osteogenic tissue or chondrogenic tissue. In various embodiments the use of potent mesenchymal stem cells discussed herein above in the manufacture of a medicament for treating bone and/or cartilage repair

According to various embodiments there is a method of treating diseases in a subject in need of Mesenchymal stem cells (MSCs) comprising modifying Mesenchymal stem cells (MSCs) to overexpress HOXA9 and administering the modified MSCs to the subject in need.

In various embodiments the modified MSCs are further modified to express a HOXA9 reporter comprising a fluorescence tag protein driven by a HOXA9 promoter. In various embodiments the fluorescence tag protein comprises an enhanced green fluorescence protein (eGFP).

In various embodiments the cells are modified with a vector.

In various embodiments the vector comprises a lentiviral system.

In various embodiments the subject is in need of bone and cartilage repair. In various embodiments the subject is in need of regenerative repair of adipogenenic tissue, osteogenic tissue or chondrogenic tissue.

### Examples

1. HOXA9 is Specifically Expressed in iPSC-MSCs and Bone Marrow-MSCs.

Using a unique and step-wise iPSC-MSC platform which recapitulated developmental stages of MSCs [FIG. 1a], gene expression profile during MSC development was compared using microarray and generated the gene list specifically expressed in both iPS-MSCs and bone marrow-MSCs, but not expressed in other stage of cells such as human fibroblasts, primitive streak and mesoderm. Microarray data showed that HOXA9 is one of top transcription factors specifically expressed in both iPS-MSCs and bone marrow-MSCs, QPCR data were consistent with microarray data [FIG. 1b].

### 2. HOXA9 Knockdown Abolished the Proliferation of MSCs

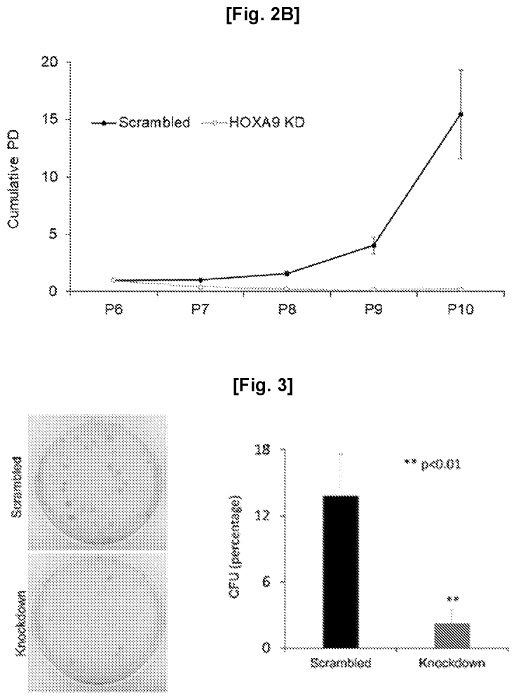

To determine the role of HOXA9 in the MSCs, MSCs were infected by lentiviral shRNA against HOXA9. QPCR and immunofluorescence showed HOXA9 was knocked down [FIG. 2a]. The effects of HOXA9 knockdown on the proliferation of MSCs was examined.

Compared to scrambled MSCs which proliferated well the knockdown MSCs lose ability to proliferate [FIG. 2b].

### 3. HOXA9 Knockdown Greatly Decreased the CFU-F of MSCs

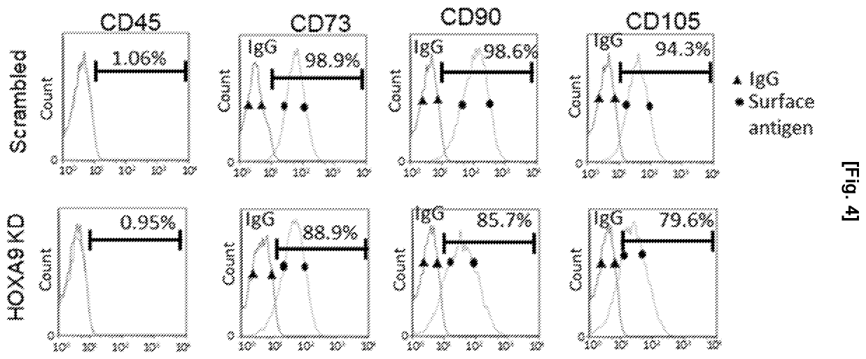

CFU-F is one important property of MSCs. Compared with scrambled control, HOXA9 knockdown greatly decreased CFU-F of MSCs [FIG. 3].

### 4. HOXA9 Knockdown Decreased the Expression of MSC Surface Antigens

According to ISCT minimal criteria, MSCs express CD73, CD90 and CD105. To determine the effects of HOXA9 knockdown on MSC surface antigen profile, FACS was performed. FACS data showed that HOXA9 knockdown decreased the percentage of CD73+, CD90+ and CD105+MSCs by at least 10% compared with scrambled control [FIG. 4].

### 5. HOXA9 Knockdown Promoted the Senescence of MSCs

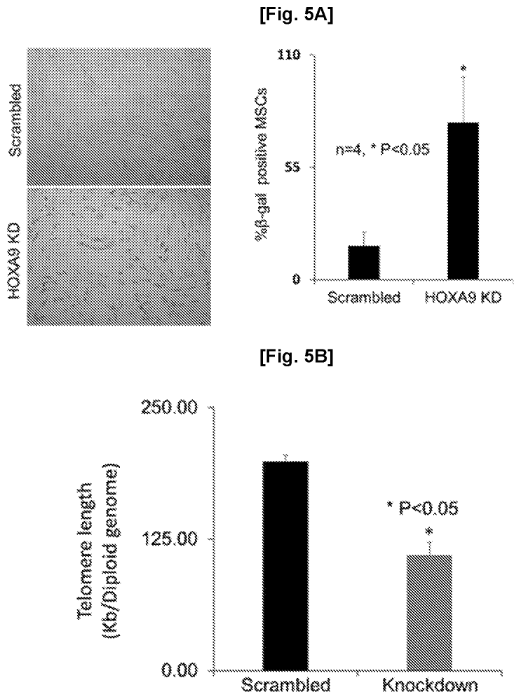

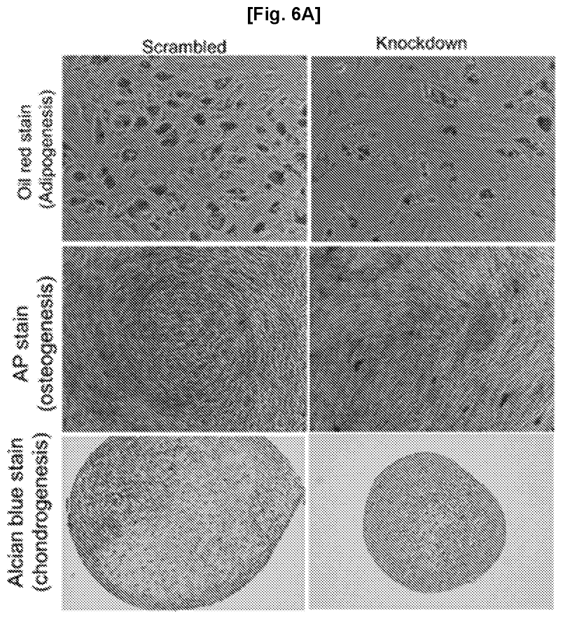

Since HOXA9 knockdown abolished the ability of MSC proliferation, the effects of HOXA9 knockdown on MSC senescence was determined. β-gal stain showed that scrambled MSCs were negative where most HOXA9 knockdown MSCs were positive for β-gal stain [FIG. 5A], showing that HOXA9 knockdown promoted the senescence of MSCs. To validate this, Changes in telomere length before and after knockdown were determined, compared with scrambled control, HOXA9 knockdown decreased the telomere length of MSCs [FIG. 5B].

### 6. HOXA9 Knockdown Slowed Down the Differentiation Potential of MSCs

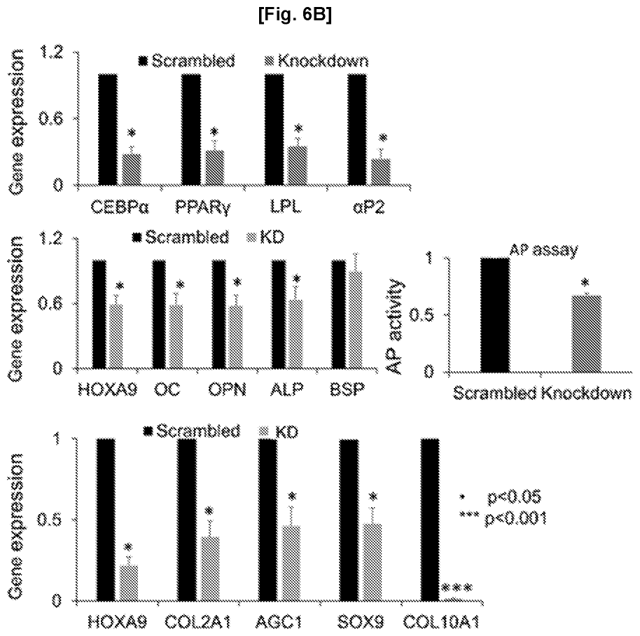

Differentiation potential is the most important property of MSCs. To determine the effects of HOXA9 knockdown on the differentiation potential of MSCs into three lineages, HOXA9 knockdown and scrambled MSCs were induced into three lineages of differentiation. Compared with scrambled control, HOXA9 knockdown decreased oil red stain for oil droplet in adipogenesis, AP stain for alkaline phosphatase in osteogenesis and alcian blue stain for proteoglycan in chondrogenesis [FIG. 6a]. Stain data were validated by qPCR for lineage specific genes, compared with scrambled control, HOXA9 knockdown decreased adipogenenic genes (CEBPα, PPARγ, LPL and αP2), osteogenic genes (OC, OPN and ALP) and chondrogenic genes (COL2A1, AGC1, SOX9 and COL10A1) [FIG. 6b]. AP assay for quantifying alkaline phosphatase activity was consistent with AP stain, HOXA9 knockdown decreased alkaline phosphatase activity [FIG. 6b]. These data showed that HOXA9 knockdown slowed down three lineages of differentiation of MSCs.

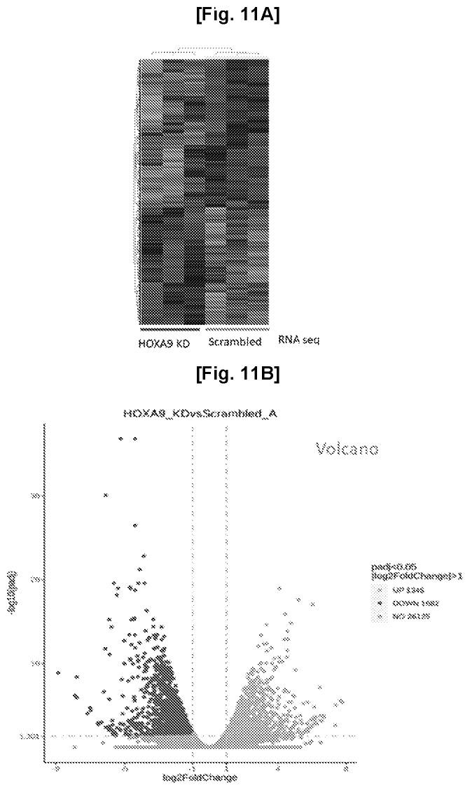

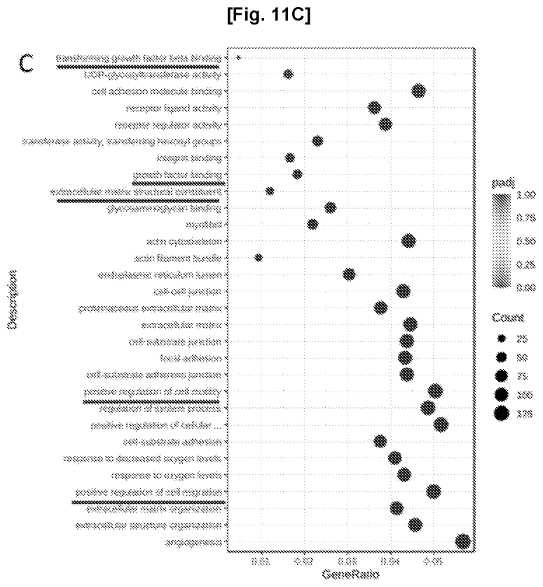

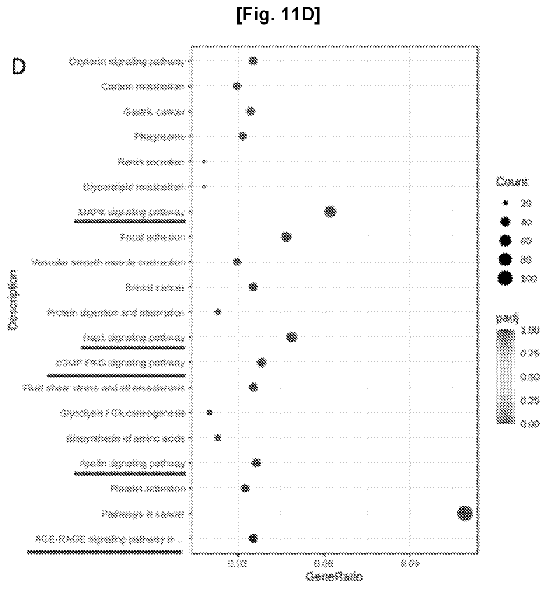

HOXA9 knockdown changed gene expression profile of MSCs (see [FIG. 11]). Compared with control, 1345 genes were upregulated whereas 1682 genes were downregulated in knockdown MSCs (see [FIG. 11B]). GO analysis showed that HOXA9 knockdown affected genes associated with transforming growth factor beta binding, growth factor binding, extracellular matrix structural constituent, and positive regulation of cell motility and migration (see [FIG. 11C]). KEGG analysis showed that HOXA9 is associated with MAPK, Rap1, cGMP-PKG, Apelin and AGE-RAGE signalling pathways (see [FIG. 11D]).

### 7. Effects of HOXA9 Specific Inhibitor DB818 on MSCs

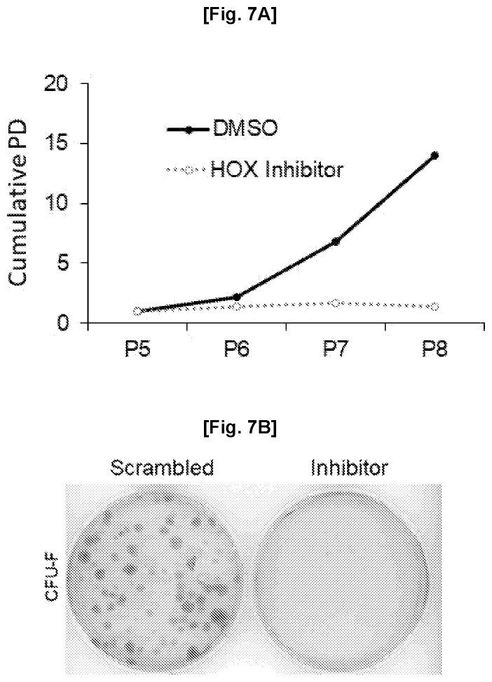

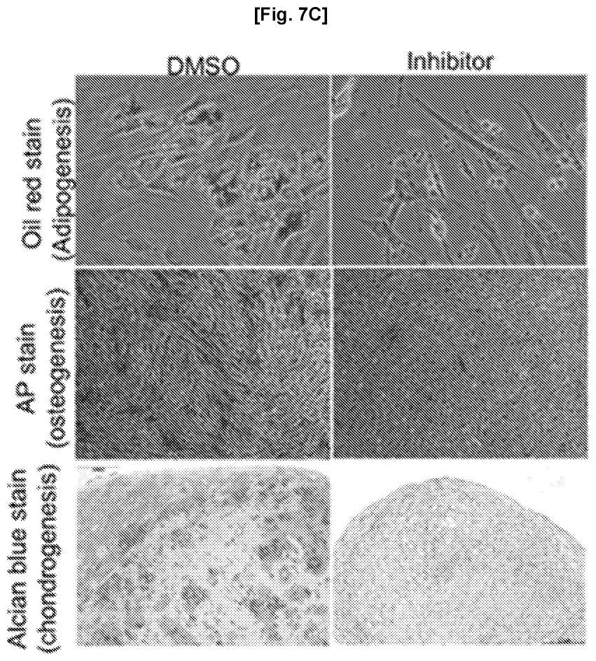

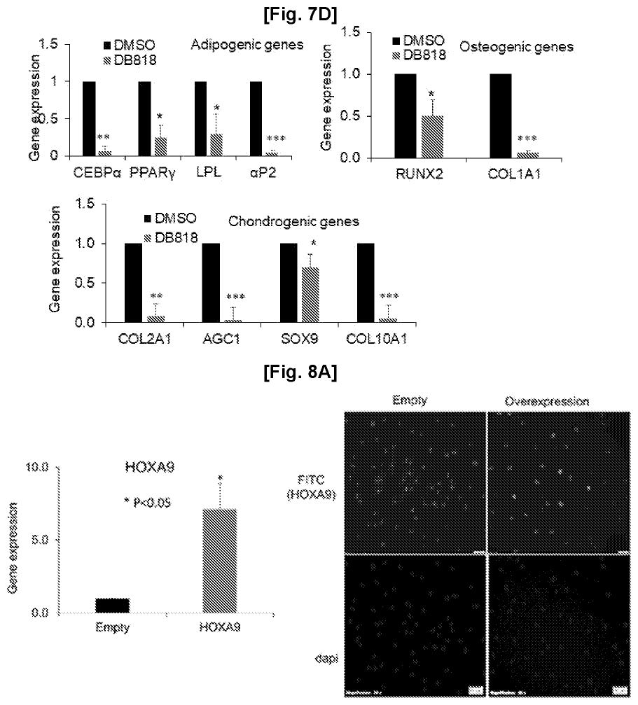

To validate the effects of HOXA9 knockdown on MSCs, effects of HOXA9 specific inhibitor DB818 on MSCs were determined. Compared with DMSO control, treatment with HOXA9 specific inhibitor DB818 abolished MSC proliferation and CFU-F. Most importantly, HOXA9 specific inhibitor DB818 also decreased oil red stain for oil droplet in adipogenesis, AP stain for alkaline phosphatase in osteogenesis and alcian blue stain for proteoglycan in chondrogenesis. Compared with scrambled control, HOXA9 inhibitor also decreased adipogenenic genes (CEBPα, PPARγ, LPL and αP2), osteogenic genes (RUNX2 and COL1A1) and chondrogenic genes (COL2A1, AGC1, SOX9 and COL10A1) [FIG. 7]. These data showed that HOXA9 inhibitor had similar effects to HOXA9 knockdown on MSC self-renewal and differentiation.

### 8. HOXA9 Overexpression Improved the Differentiation Potential of MSCs

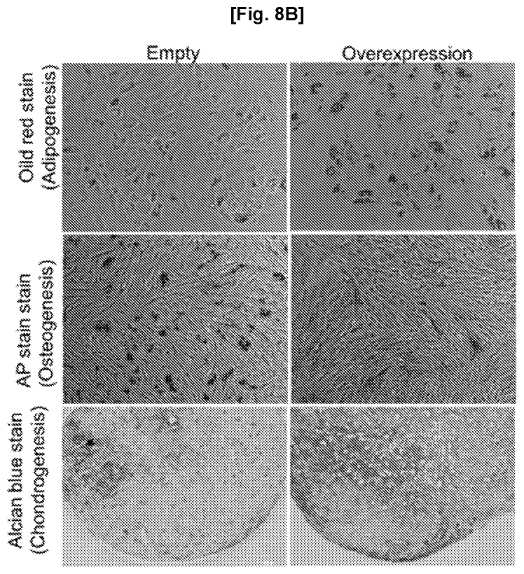

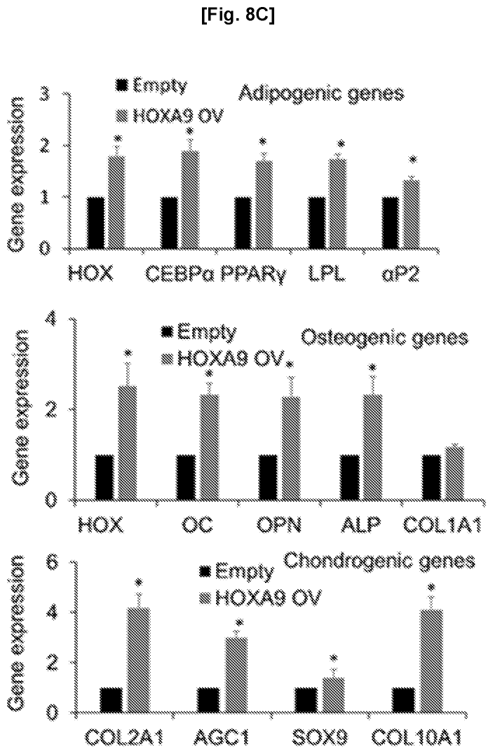

To determine the effects of HOXA9 overexpression on differentiation potential of MSCs, HOXA9 was overexpressed with lentiviral system [FIG. 8A]. Compared with empty control, HOXA9 overexpression improved oil red stain for oil droplet in adipogenesis, AP stain for alkaline phosphatase in osteogenesis and alcian blue stain for proteoglycan in chondrogenesis. Stain data were validated by qPCR for lineage specific gene. HOXA9 overexpression consistently increased adipogenenic genes (CEBPα, PPARγ, LPL and αP2), osteogenic genes (OC, OPN and ALP) and chondrogenic genes (COL2A1, AGC1, SOX9 and COL10A1) (FIG. 8B). These data showed that HOXA9 overexpression improved three lineages of differentiation of MSCs.

### 9. Molecular Basis of HOXA9 Regulating MSC Stemness/Potency

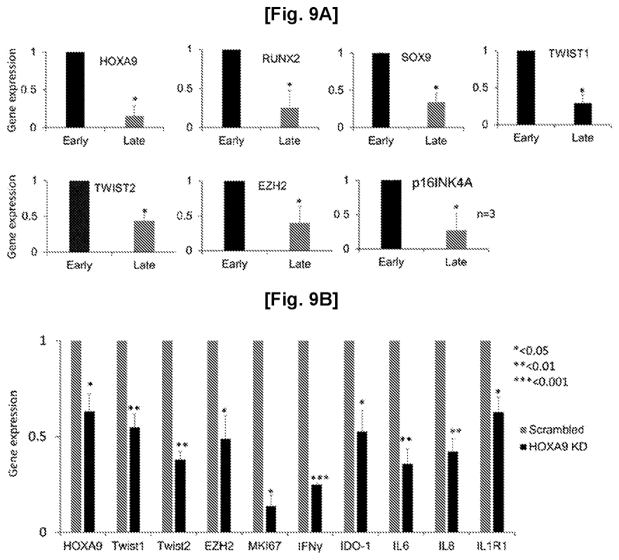

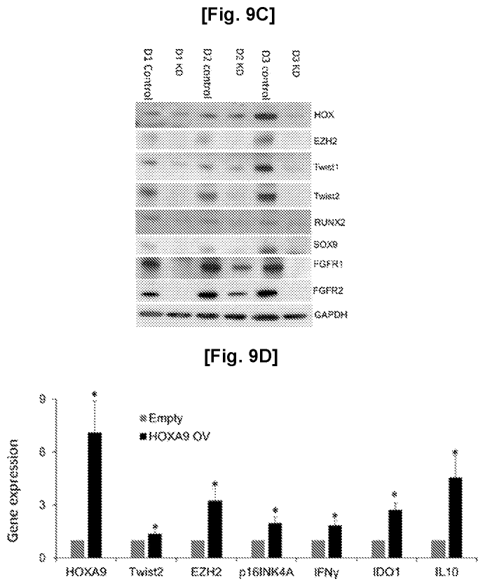

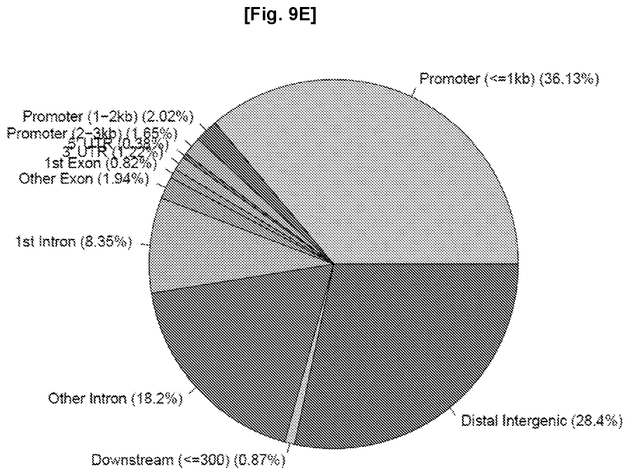

To understand the molecular basis of HOXA9 regulating MSC stemness/potency, HOXA9 expression in late passage of MSCs versus early passage of MSCs was determined. Compared with early passage of MSCs, HOXA9 expression in late passage of MSCs was much decreased. Most importantly, HOXA9 expression correlated with MSC important genes such as RUNX2, SOX9, TWIST1, TWIST2, EZH2 and p16INKA4A [FIG. 9A]. Compared with scrambled control, HOXA9 knockdown decreased MSC important genes, including TWIST1, TWIST2, EZH2 as well as secreting genes IFNγ, IDO1, IL6, IL8, etc [FIG. 9B]. To validate qPCR data, western blotting was performed, which was consistent with qPCR data, showing that HOXA9 knockdown decreased the expression of MSC important proteins such as RUNX2, SOX9, TWIST1, TWIST2, EZH2 [FIG. 9C]. In contrast, HOXA9 overexpression increased the expression of TWIST2 and EZH2 [FIG. 9D].

To understand which genes were directly regulated by HOXA9, chromatin immunoprecipitation sequencing (ChIP-seq) against HOXA9 was performed. ChIP-seq data showed that HOXA9 most bound to the promoter and distal intergenic regions of target genes [FIG. 9E]. The direct target genes of HOXA9 were associated with cellular senescence, cell cycle, FOXO signalling pathway and hippo signalling pathway [FIG. 9F].

### 10. HOXA9 Overexpression Did not Cause Tumorigenesis

To test whether HOXA9 overexpression causes tumorigenesis, HOXA9-overexpressing MSCs were subcutaneously transplanted into immunodeficient mice, after 12 weeks of transplant, no tumour was observed, suggesting HOXA9 overexpression did not cause tumorigenesis (data not shown).

11. Distinguishing MSC from Human Fibroblasts

HOXA9 can be used to distinguish human MSCs from human fibroblasts, both are very similar. The former highly express HOXA9 whereas the latter express very low or no HOXA9.

### 12. The Use of HOXA9 Reporter to Enrich Good Quality of MSCs.

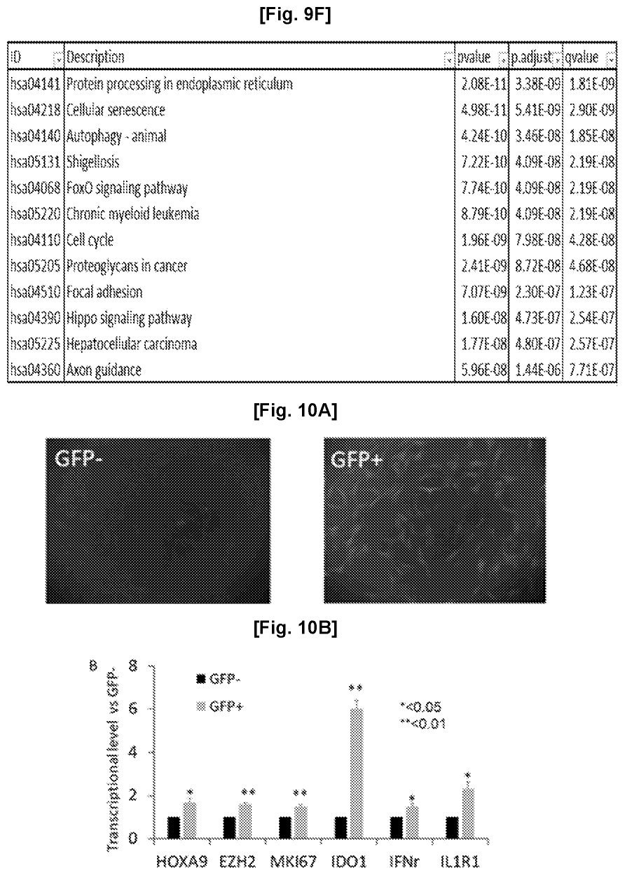

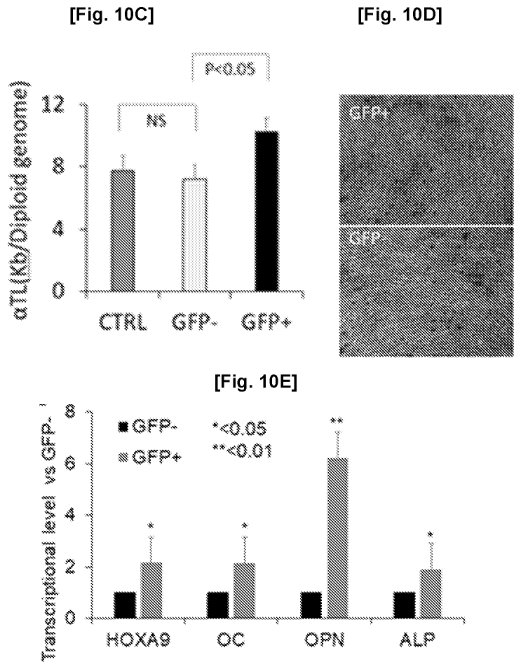

In [FIG. 10] immortalized MSCs (UE7T-13) were infected with lentiviral HOXA9 reporter consisting of HOXA9 promoter driven eGFP, the strongest and the weakest eGFP cells were sorted based on fluorescence expression (see [FIG. 10A]). The strong reporter MSCs identified by this method expressed higher levels of EZH2, MKI67, IDO1, IFNγ and IL1R1 than weak reporter MSCs (see [FIG. 10B]). Similarly, the strong reporter MSCs identified by this method have longer telomere length than the weak reporter MSCs identified by this method (see [FIG. 10C]). Upon osteogenesis for 2 weeks, the strong reporter MSCs identified by this method accumulated more calcium deposit shown by alizarin red stain (see [FIG. 10D]) and express higher osteogenic genes such as OC, OPN and ALP than weak reporter MSCs identified by this method (see [FIG. 10E]).

### 13. Investigation of how HOXA9 Regulates MSC Stemness

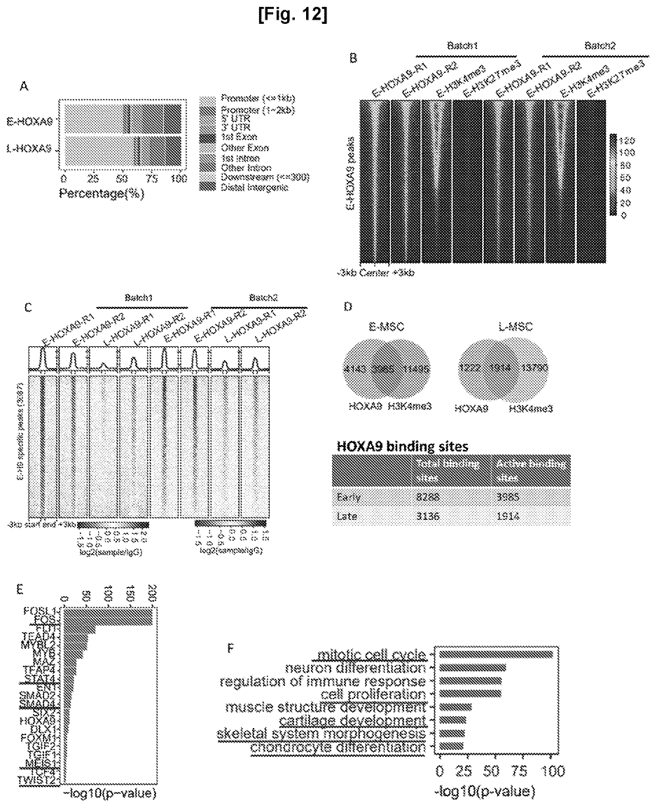

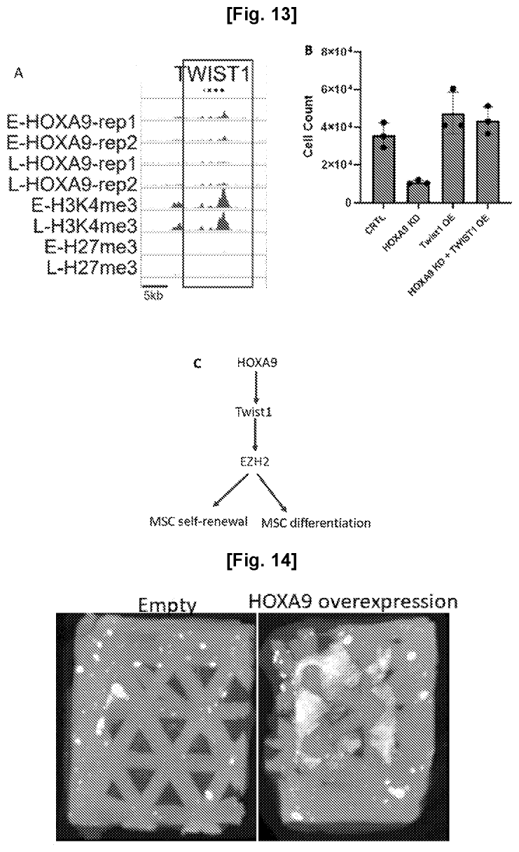

To understand how HOXA9 regulates MSC stemness, HOXA9 binding sites at early and late passage of MSCs were determined by chromatin immunoprecipitation assays with sequencing (ChIP-seq). HOXA9 predominantly binds to the promoters (see [FIG. 12A]). HOXA9 binding sites associate with H3K4me3 binding sites for active genes but are depleted in H3K4me27 binding sites for inactive genes (see [FIG. 12B]). Most importantly, HOXA9 binding sites decreases more at late passage of MSCs compared with early passage of MSCs (see [FIG. 12C]), this maybe associates with the loss of MSC stemness at late passage. Total binding sites decreased from 8288 at early passage of MSCs to 3136 at late passage of MSCs, active binding sites decreased from 3985 at early passage of MSCs to 1914 at late passage of MSCs (see [FIG. 12D]). HOXA9 binding sites overlapped with FOS, STAT4, SMAD4, MEIS1 and TWIST2 binding sites (see [FIG. 12E]). HOXA9 binding sites are also associated with mitotic cell cycle, cell proliferation, cartilage development, skeletal system morphogenesis and chondrocytes (see [FIG. 12F]). All of this suggests that HOXA9 regulates MSC stemness by binding to self-renewal and differentiation gene of MSCs.

ChIP-seq data showed that HOXA9 directly binds to TWIST1 (see [FIG. 13A]), the important MSC gene associated with MSC self-renewal and differentiation. Rescue experiment showed that HOXA9 knockdown greatly decreased MSC proliferation whereas TWIST1 overexpression increased MSC proliferation (see [FIG. 13B]). Most importantly, TWIST1 overexpression can rescue the effects of HOXA9 knockdown on MSC proliferation (see [FIG. 13B]). Based on ChIP-seq, HOXA9 knockdown, inhibitor and overexpression data, the mechanism of HOXA9 regulating MSC stemness is proposed, HOXA9 directly binds to TWIST1, which Induces EZH2 recruitment regulating histone methylation to regulate MSC self-renewal and differentiation potential (see [FIG. 13C]).

### 14. Using Potent MSCs Modified to Overexpress HOXA9 In Vivo to Improve Bone Repair.

To test if HOXA9 overexpression improves bone formation in vivo, HOXA9 overexpressing and empty control MSCs were seeded onto the bone scaffold of PCL-TCP with help of fibrin glue, after 1 week of pre-differentiation in osteogenic medium, differentiated cells were subcutaneously transplanted into immunodeficient mice for 8 weeks. μCT data showed that HOXA9 overexpression significantly improved bone formation in vivo compared with empty control.

### STATEMENTS OF INVENTION

1. A method of identifying therapeutically potent Mesenchymal stem cells (MSCs) comprising detecting HOXA9 expression; wherein an expression value at or over a first predetermined value indicates the MSCs are therapeutically potent.
2. A method of distinguishing Mesenchymal stem cells (MSCs) from human fibroblasts comprising detecting HOXA9 expression in the cell; wherein an expression value at or over a second predetermined value indicates the cell is an MSCs and an expression value below the second predetermined value indicates the cell is a human fibroblast.
3. The method according to statement 1 or 2, further comprising
introducing a HOXA9 reporter into the cells wherein the HOXA9 reporter comprises a fluorescence tag protein driven by a HOXA9 promoter;
incubating the cells; and
measuring the fluorescence of the HOXA9 reporter to determine the equivalent of detecting HOXA9 expression at or over the first predetermined value or the second predetermined level.
4. The method according to statement 3, wherein MSC's are sorted into MSCs expressing the HOXA9 reporter at or over the first predetermined value or the second predetermined level.
5. The method according to any one of statements 1 to 4, wherein MSC's are sorted into MSCs expressing markers selected from any one of EZH2, MIK67, IDO1 IFNγ and a combination thereof.
6. The method according to any one of statements 2 to 5, wherein the MSCs expressing HOXA9 at or over the second predetermined value expressed at least one gene selected from the group consisting of RUNX2, SOX9, TWIST1, TWIST2, EZH2 and p16INKA4A at an increased amount compared to the expression of at least one the genes in MSCs expressing HOXA9 below the second predetermined value.
7. Potent Mesenchymal stem cells (MSCs) comprising stem cells modified to overexpress HOXA9, wherein the modified cells demonstrate adipogenenic, osteogenic and chondrogenic differentiation potential.
8. The Potent Mesenchymal stem cells according to statement 7, wherein the cells are further modified to express a HOXA9 reporter comprising a fluorescence tag protein driven by a HOXA9 promoter.
9. The Potent Mesenchymal stem cells according to statement 7 or 8, wherein expression of TWIST2 and EZH2 was increased compared to the expression of these genes in MSCs that are not modified to overexpress HOXA9.
10. The Potent Mesenchymal stem cells according to any one of statements 7 to 9, wherein the modified cells express at least one gene selected from the group consisting of CEBPα, PPARγ, LPL, αP2, OC, OPN, ALP, COL2A1, AGC1, SOX9 and COL10A1.
11. The Potent Mesenchymal stem cells according to any one of statements 7 to 9, wherein expression of adipogenenic genes CEBPα, PPARγ, LPL and αP2 was higher than the expression of these genes in MSCs that are not modified to overexpress HOXA9.
12. The Potent Mesenchymal stem cells according to any one of statements 7 to 9, wherein expression of osteogenic genes OC, OPN and ALP was higher than the expression of these genes in MSCs that are not modified to overexpress HOXA9.
13. The Potent Mesenchymal stem cells according to any one of statements 7 to 9, wherein expression of chondrogenic genes COL2A1, AGC1, SOX9 and COL10A1 was higher than the expression of these genes in MSCs that are not modified to overexpress HOXA9.
14. The Potent Mesenchymal stem cells according to any one of statement 7-13 for use in treating a disease.
15. The cells for use according to statement 14, wherein the disease comprises bone and/or cartilage repair.
16. A method of treating diseases in a subject in need of Mesenchymal stem cells (MSCs) comprising modifying Mesenchymal stem cells (MSCs) to overexpress HOXA9 and administering the modified MSCs to the subject in need.
17. The method according to statement 16, wherein the modified MSCs are further modified to express a HOXA9 reporter comprising a fluorescence tag protein driven by a HOXA9 promoter.
18. The method according to statement 16, wherein the cells are modified with a vector.
19. The method according to statement 17, wherein the cells are modified with a vector.
20. The method according to statement 18, wherein the vector comprises a lentiviral system.
21. The method according to statement 16, wherein the subject is in need of bone and cartilage repair.

It should be further appreciated by the person skilled in the art that variations and combinations of features described above, not being alternatives or substitutes, may be combined to form yet further embodiments falling within the intended scope of the invention.

As would be understood by a person skilled in the art, each embodiment, may be used in combination with other embodiment or several embodiments.

1. A method of identifying therapeutically potent Mesenchymal stem cells (MSCs) comprising detecting HOXA9 expression; wherein an expression value at or over a first predetermined value indicates the MSCs are therapeutically potent.

2. A method of distinguishing Mesenchymal stem cells (MSCs) from human fibroblasts comprising detecting HOXA9 expression in the cell; wherein an expression value at or over a second predetermined value indicates the cell is an MSCs and an expression value below the second predetermined value indicates the cell is a human fibroblast.

3. The method according to claim 1, further comprising,
introducing a HOXA9 reporter into the cells wherein the HOXA9 reporter comprises a fluorescence tag protein driven by a HOXA9 promoter;
incubating the cells; and
measuring the fluorescence of the HOXA9 reporter to determine the equivalent of detecting HOXA9 expression at or over the first predetermined value or the second predetermined level.

4. The method according to claim 3, wherein MSC's are sorted into MSCs expressing the HOXA9 reporter at or over the first predetermined value or the second predetermined level.

5. The method according to claim 1, wherein MSC's are sorted into MSCs expressing markers selected from any one of EZH2, MIK67, IDO1 IFNγ and a combination thereof.

6. The method according to claim 2, wherein the MSCs expressing HOXA9 at or over the second predetermined value expressed at least one gene selected from the group consisting of RUNX2, SOX9, TWIST1, TWIST2, EZH2 and p16INKA4A at an increased amount compared to the expression of at least one the genes in MSCs expressing HOXA9 below the second predetermined value.

7. Potent Mesenchymal stem cells (MSCs) comprising stem cells modified to overexpress HOXA9, wherein the modified cells demonstrate adipogenenic, osteogenic and chondrogenic differentiation potential.

8. The Potent Mesenchymal stem cells according to claim 7, wherein the cells are further modified to express a HOXA9 reporter comprising a fluorescence tag protein driven by a HOXA9 promoter.

9. The Potent Mesenchymal stem cells according to claim 7, wherein expression of TWIST2 and EZH2 was increased compared to the expression of these genes in MSCs that are not modified to overexpress HOXA9.

10. The Potent Mesenchymal stem cells according to claim 7, wherein the modified cells express at least one gene selected from the group consisting of CEBPα, PPARγ, LPL, αP2, OC, OPN, ALP, COL2A1, AGC1, SOX9 and COL10A1.

11. The Potent Mesenchymal stem cells according to claim 7, wherein expression of adipogenenic genes CEBPα, PPARγ, LPL and αP2 was higher than the expression of these genes in MSCs that are not modified to overexpress HOXA9.

12. The Potent Mesenchymal stem cells according to claim 7, wherein expression of osteogenic genes OC, OPN and ALP was higher than the expression of these genes in MSCs that are not modified to overexpress HOXA9.

13. The Potent Mesenchymal stem cells according to claim 7, wherein expression of chondrogenic genes COL2A1, AGC1, SOX9 and COL10A1 was higher than the expression of these genes in MSCs that are not modified to overexpress HOXA9.

14.-15. (canceled)

16. A method of treating diseases in a subject in need of Mesenchymal stem cells (MSCs) comprising modifying Mesenchymal stem cells (MSCs) to overexpress HOXA9 and administering the modified MSCs to the subject in need.

17. The method according to claim 16, wherein the modified MSCs are further modified to express a HOXA9 reporter comprising a fluorescence tag protein driven by a HOXA9 promoter.

18. The method according to claim 16, wherein the cells are modified with a vector.

19. The method according to claim 16, wherein the cells are modified with a vector comprising a lentiviral system.

20. The method according to claim 16, wherein the subject is in need of bone and cartilage repair.


SEQUENCE LISTING (24 sequences)

SEQ ID NO: 1 - DNA, 3100 bp, Homo sapiens
  source 1..3100: mol_type = genomic DNA
gccaccaagttgttacatgaaatctgcagtttcataatttccgtgggtcgggccgggcgggccaggcgctgggcacggtgatggccaccactggggccctgggcaactactacgtggactcgttcctgctgggcgccgacgccgcggatgagctgagcgttggccgctatgcgccggggaccctgggccagcctccccggcaggcggcgacgctggccgagcaccccgacttcagcccgtgcagcttccagtccaaggcgacggtgtttggcgcctcgtggaacccagtgcacgcggcgggcgccaacgctgtacccgctgcggtgtaccaccaccatcaccaccacccctacgtgcacccccaggcgcccgtggcggcggcggcgccggacggcaggtacatgcgctcctggctggagcccacgcccggtgcgctctccttcgcgggcttgccctccagccggccttatggcattaaacctgaaccgctgtcggccagaaggggtgactgtcccacgcttgacactcacactttgtccctgactgactatgcttgtggttctcctccagttgatagagaaaaacaacccagcgaaggcgccttctctgaaaacaatgctgagaatgagagcggcggagacaagccccccatcgatcccagtaagtgtctcctcccttcaaatccgccgccgcctccacgccggcctcccggatctgctggcccgccaggtttctctcgagcctgccttcgtcctcgctggaagcctctcgagttggggccaggagccagaagttggtgtttgggacgcctcagatagggccccaagtctggagagcagtgaagagcggcccgcagggctacgggagaggaggcggctgctgcagcgagagggggcggggcgggcacttcgggacgagccaagactggccgcccctctccttggctgcccaggcccaggaccgagatactttgggccgttcttcgaaagcagtgcagcccagagagccttttgtacaactagattgtccgtgagcggcggcagccagggcagccggagctgggacgctgggggagacggccgattccttccacttcttgccttcggccagtggcggcgtaaatcctgccaagatgaggctgcgggcgacccgggccacaagggtccccatgacagattattcaaataagccacagacgtgatcagcggccttagggcgccctgacggcttgcccagctccgaaggccttccaggaaggttaaataaggagtggggggcgtagagggacaggttgggaaagaaagacgaagtcagtgaacgggacagaggaatcctaatcttgctacagaacacaaggcagcatgctttccctctgcgtggcaaggagacctgtttccaaatttcattctatacagcgttttgagagtgggaggaaggagaagggggacaagaggacagagtaggagaaaggaaggtctcggaggggagggcgagccaaagttttactgcgtgcaattttaagtgactgtctgtgcgtctgtctgccagggttccattgtgtccgaggcctgactgcctttcctaaccagttcagcagagttctgcacttcggccagagaccccatgcaggaggctcatttgccccagcgggatgtgcgtcttctgctcctaaacccagtgtttctcttccccgcagataacccagcagccaactggcttcatgcgcgctccactcggaaaaagcggtgcccctatacaaaacaccagaccctggaactggagaaagagtttctgttcaacatgtacctcaccagggaccgcaggtacgaggtggctcgactgctcaacctcaccgagaggcaggtcaagatctggttccagaaccgcaggatgaaaatgaagaaaatcaacaaagaccgagcaaaagacgagtgatgccatttgggcttatttagaaaaaagggtaagctagagagaaaaagaaagaactgtccgtcccccttccgccttctcccttctctcacccccaccctagcctccaccatccccgcacaaagcggctctaaacctcaggccacatcttttccaaggcaaaccctgttcaggctggctcgtaggcctgccgctttgatggaggaggtattgtaagctttccattttctataagaaaaaggaaaagttgaggggggggcattagtgctgatagctgtgtgtgttagcttgtatatatatttttaaaaatctacctgttcctgacttaaaacaaaaggaaagaaactacctttttataatgcacaactgttgatggtaggctgtatagtttttagtctgtgtagttaatttaatttgcagtttgtgcggcagattgctctgccaagatacttgaacactgtgttttattgtggtaattatgttttgtgattcaaacttctgtgtactgggtgatgcacccattgtgattgtggaagatagaattcaatttgaactcaggttgtttatgaggggaaaaaaacagttgcatagagtatagctctgtagtggaatatgtcttctgtataactaggctgttaacctatgattgtaaagtagctgtaagaatttcccagtgaaataaaaaaaaattttaagtgttctcggggatgcatagattcatcattttctccaccttaaaaatgcgggcatttaagtctgtccattatctatatagtcctgtcttgtctattgtatatataatctatatgattaaagaaaatatgcataatcagacaagcttgaatattgtttttgcaccagacgaacagtgaggaaattcggagctatacatatgtgcagaaggttactacctagggtttatgcttaattttaattggaggaaatgaatgctgattgtaacggagttaattttattgataataaattatacactatgaaaccgccattgggctactgtagatttgtatccttgatgaatctggggtttccatcagactgaacttacactgtatattttgcaatagttacctcaaggcctactgaccaaattgttgtgttgagatgatatttaactttttgccaaataaaatatattgattcttttcta

SEQ ID NO: 2 - DNA, 1276 bp, Homo sapiens
  source 1..1276: mol_type = genomic DNA
gccccgggacaggccacatcggtgcgggcctcccaggttccggagctgcggggtctcttaggcgaggctgccttttcccaaaccgaacttgccttccattcatgccacttgtagttttttccccagctgggattcacggagcgcaaccaggcttgcagcgctcatggttagagcctctgaggctggagcacagggctgggtcgccagccgcctgcgcctgggaatcctgattgccagctgatgagaaaggcgggctgggcgcgcgtgtgcgtggggtcgagggccggggaccgagcgcgccgcacaaccaaccaggccctcaaaaccttcgccctggtggcggctggccgctccctcctggccagctcctccgtggggtcctcgtagcaaaggcgaatttaagggttgcccgggcgcccctcgctccaggcgggtagctgtggggacctacacccgcggtactccctgagcggccggtccctgcctggagtgccctggtagggccggcggcggctccgtttgggacggatcctgcgttgaatttgacttttcgagggcggccgcgggtaaactcgcctctcccggggaccgcagggattatttacagggagctcgccaaccaaacacaacagtctaacctttccaagtcctcgtaaatttttacagctgggagccacggcgaggcaaacgaatctgttggtcgtttccgacttcccgccagcctgtgtggcttctgaaacaataactccttatgaaatatcataaatatagatttaaatacagtagagcgacaatgcgatttggctgcttttttatggcttcaattattgtctaattttatgtgaggggctccgctggccgcactcgcacgcgggacccgcgccttcttgatggcgtgattaattgtgatataaaatagtccgcttaagaagtgtgtgtatggggggggagacgggagagtacagagacaaggctagatttgatcttttaatcgtcgttggccacaattaaaacaaaccccatcgtagagcggcacgatccctttacataaaaacatatggcttttgctataaaaattatgactgcaaaacatcggaccattaatagcgtgcggagtgatttacgcgttattgttctgctggacgggcacgtgacgcgcacggccaatgggggcgcgggcgccggcaacttattaggtgactgtacttcccccccggtgccaccaagttgttacatgaaatctgcagtttcataatttccgtgggtcgggccgggc

SEQ ID NO: 3 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 4 - protein, 272 aa, Homo sapiens [sequence omitted]

SEQ ID NO: 5 - RNA, 22 nt, synthetic construct
  source 1..22: mol_type = other RNA
agccggccttatggcattaaac

SEQ ID NO: 6 - RNA, 22 nt, synthetic construct
  source 1..22: mol_type = other RNA
ttctcctccagttgatagagaa

SEQ ID NO: 7 - RNA, 21 nt, synthetic construct
  source 1..21: mol_type = other RNA
cctaaggttaagtcgccctcg

SEQ ID NO: 8 - protein, 521 aa, Homo sapiens
MASNSLFSTVTPCQQNFFWDPSTSRRFSPPSSSLQPGKMSDVSPVVAAQQQQQQQQQQQQQQQQQQQQQQQEAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRGKSFTLTITVFTNPPQVATYHRAIKVTVDGPREPRRHRQKLDDSKPSLFSDRLSDLGRIPHPSMRVGVPPQNPRPSLNSAPSPFNPQGQSQITDPRQAQSSPPWSYDQSYPSYLSQMTSPSIHSTTPLSSTRGTGLPAITDVPRRISDDDTATSDFCLWPSTLSKKSQAGASELGPFSDPRQFPSISSLTESRFSNPRMHYPATFTYTPPVTSGMSLGMSATTHYHTYLPPPYPGSSQSQSGPFQTSSTPYLYYGTSSGSYQFPMVPGGDRSPSRMLPPCTTTSNGSTLLNPNLPNQNDGVDADGSHSSSPTVLNSSGRMDESVWRPY

SEQ ID NO: 9 - protein, 509 aa, Homo sapiens
MNLLDPFMKMTDEQEKGLSGAPSPTMSEDSAGSPCPSGSGSDTENTRPQENTFPKGEPDLKKESEEDKFPVCIREAVSQVLKGYDWTLVPMPVRVNGSSKNKPHVKRPMNAFMVWAQAARRKLADQYPHLHNAELSKTLGKLWRLLNESEKRPFVEEAERLRVQHKKDHPDYKYQPRRRKSVKNGQAEAEEATEQTHISPNAIFKALQADSPHSSSGMSEVHSPGEHSGQSQGPPTPPTTPKTDVQPGKADLKREGRPLPEGGRQPPIDFRDVDIGELSSDVISNIETFDVNEFDQYLPPNGHPGVPATHGQVTYTGSYGISSTAATPASAGHVWMSKQQAPPPPPQQPPQAPPAPQAPPQPQAAPPQQPAAPPQQPQAHTLTTLSSEPGQSQRTHIKTEQLSPSHYSEQQQHSPQQIAYSPFNLPHYSPSYPPITRSQYDYTDHQNSSSYYSHAAGQGTGLYSTFTYMNPAQRPMYTPIADTSGVPSIPQTHSPQHWEQPVYTQLTRP

SEQ ID NO: 10 - protein, 202 aa, Homo sapiens
MMQDVSSSPVSPADDSLSNSEEEPDRQQPPSGKRGGRKRRSSRRSAGGGAGPGGAAGGGVGGGDEPGSPAQGKRGKKSAGCGGGGGAGGGGGSSSGGGSPQSYEELQTQRVMANVRERQRTQSLNEAFAALRKIIPTLPSDKLSKIQTLKLAARYIDFLYQVLQSDELDSKMASCSYVAHERLSYAFSVWRMEGAWSMSASH

SEQ ID NO: 11 - protein, 160 aa, Homo sapiens
MEEGSSSPVSPVDSLGTSEEELERQPKRFGRKRRYSKKSSEDGSPTPGKRGKKGSPSAQSFEELQSQRILANVRERQRTQSLNEAFAALRKIIPTLPSDKLSKIQTLKLAARYIDFLYQVLQSDEMDNKMTSCSYVAHERLSYAFSVWRMEGAWSMSASH

SEQ ID NO: 12 - protein, 746 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 13 - protein, 132 aa, Homo sapiens
MVRRFLVTLRIRRACGPPRVRVFVVHIPRLTGEWAAPGAPAAVALVLMLLRSQRLGQQPLPRRPGHDDGQRPSGGAAAAPRRGAQLRRPRHSHPTRARRCPGGLPGHAGGAAPGRGAAGRARCLGPSARGPG

SEQ ID NO: 14 - protein, 156 aa, Homo sapiens
MEPAAGSSMEPSADWLATAAARGRVEEVRALLEAGALPNAPNSYGRRPIQVMMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPD

SEQ ID NO: 15 - protein, 358 aa, Homo sapiens
MESADFYEAEPRPPMSSHLQSPPHAPSSAAFGFPRGAGPAQPPAPPAAPEPLGGICEHETSIDISAYIDPAAFNDEFLADLFQHSRQQEKAKAAVGPTGGGGGGDFDYPGAPAGPGGAVMPGGAHGPPPGYGCAAAGYLDGRLEPLYERVGAPALRPLVIKQEPREEDEAKQLALAGLFPYQPPPPPPPSHPHPHPPPAHLAAPHLQFQIAHCGQTTMHLQPGHPTPPPTPVPSPHPAPALGAAGLPGPGSALKGLGAAHPDLRASGGSGAGKAKKSVDKNSNEYRVRRERNNIAVRKSRDKAKQRNVETQQKVLELTSDNDRLRKRVEQLSRELDTLRGIFRQLPESSLVKAMGNCA

SEQ ID NO: 16 - protein, 505 aa, Homo sapiens
MGETLGDSPIDPESDSFTDTLSANISQEMTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 17 - protein, 475 aa, Homo sapiens
MESKALLVLTLAVWLQSLTASRGGVAAADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSG

SEQ ID NO: 18 - protein, 132 aa, Homo sapiens
MCDAFVGTWKLVSSENFDDYMKEVGVGFATRKVAGMAKPNMIISVNGDVITIKSESTFKNTEISFILGQEFDEVTADDRKVKSTITLDGGVLVHVQKWDGKSTTIKRKREDDKLVVECVMKGVTSTRVYERA

SEQ ID NO: 19 - protein, 100 aa, Homo sapiens
MRALTLLALLALAALCIAGQAGAKPSGAESSKGAAFVSKQEGSEVVKRPRRYLYQWLGAPVPYPDPLEPRREVCELNPDCDELADHIGFQEAYRRFYGPV

SEQ ID NO: 20 - protein, 314 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 21 - protein, 524 aa, Homo sapiens
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAGPLLLALALYPLSVLF

SEQ ID NO: 22 - protein, 1487 aa, Homo sapiens
MIRLGAPQTLVLLTLLVAAVLRCQGQDVQEAGSCVQDGQRYNDKDVWKPEPCRICVCDTGTVLCDDIICEDVKDCLSPEIPFGECCPICPTDLATASGQPGPKGQKGEPGDIKDIVGPKGPPGPQGPAGEQGPRGDRGDKGEKGAPGPRGRDGEPGTPGNPGPPGPPGPPGPPGLGGNFAAQMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRADQAAGGLRQHDAEVDATLKSLNNQIESIRSPEGSRKNPARTCRDLKLCHPEWKSGDYWIDPNQGCTLDAMKVFCNMETGETCVYPNPANVPKKNWWSSKSKEKKHIWFGETINGGFHFSYGDDNLAPNTANVQMTFLRLLSTEGSQNITYHCKNSIAYLDEAAGNLKKALLIQGSNDVEIRAEGNSRFTYTALKDGCTKHTGKWGKTVIEYRSQKTSRLPIIDIAPMDIGGPEQEFGVDIGPVCFL

SEQ ID NO: 23 - protein, 2530 aa, Homo sapiens
MTTLLWVFVTLRVITAAVTVETSDHDNSLSVSIPQPSPLRVLLGTSLTIPCYFIDPMHPVTTAPSTAPLAPRIKWSRVSKEKEVVLLVATEGRVRVNSAYQDKVSLPNYPAIPSDATLEVQSLRSNDSGVYRCEVMHGIEDSEATLEVVVKGIVFHYRAISTRYTLDFDRAQRACLQNSAIIATPEQLQAAYEDGFHQCDAGWLADQTVRYPIHTPREGCYGDKDEFPGVRTYGIRDTNETYDVYCFAEEMEGEVFYATSPEKFTFQEAANECRRLGARLATTGQLYLAWQAGMDMCSAGWLADRSVRYPISKARPNCGGNLLGVRTVYVHANQTGYPDPSSRYDAICYTGEDFVDIPENFFGVGGEEDITVQTVTWPDMELPLPRNITEGEARGSVILTVKPIFEVSPSPLEPEEPFTFAPEIGATAFAEVENETGEATRPWGFPTPGLGPATAFTSEDLVVQVTAVPGQPHLPGGVVFHYRPGPTRYSLTFEEAQQACLRTGAVIASPEQLQAAYEAGYEQCDAGWLRDQTVRYPIVSPRTPCVGDKDSSPGVRTYGVRPSTETYDVYCFVDRLEGEVFFATRLEQFTFQEALEFCESHNATLATTGQLYAAWSRGLDKCYAGWLADGSLRYPIVTPRPACGGDKPGVRTVYLYPNQTGLPDPLSRHHAFCFRGISAVPSPGEEEGGTPTSPSGVEEWIVTQVVPGVAAVPVEEETTAVPSGETTAILEFTTEPENQTEWEPAYTPVGTSPLPGILPTWPPTGAATEESTEGPSATEVPSASEEPSPSEVPFPSEEPSPSEEPFPSVRPFPSVELFPSEEPFPSKEPSPSEEPSASEEPYTPSPPVPSWTELPSSGEESGAPDVSGDFTGSGDVSGHLDFSGQLSGDRASGLPSGDLDSSGLTSTVGSGLPVESGLPSGDEERIEWPSTPTVGELPSGAEILEGSASGVGDLSGLPSGEVLETSASGVGDLSGLPSGEVLETTAPGVEDISGLPSGEVLETTAPGVEDISGLPSGEVLETTAPGVEDISGLPSGEVLETTAPGVEDISGLPSGEVLETTAPGVEDISGLPSGEVLETTAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETTAPGVEEISGLPSGEVLETTAPGVDEISGLPSGEVLETTAPGVEEISGLPSGEVLETSTSAVGDLSGLPSGGEVLEISVSGVEDISGLPSGEVVETSASGIEDVSELPSGEGLETSASGVEDLSRLPSGEEVLEISASGFGDLSGLPSGGEGLETSASEVGTDLSGLPSGREGLETSASGAEDLSGLPSGKEDLVGSASGDLDLGKLPSGTLGSGQAPETSGLPSGFSGEYSGVDLGSGPPSGLPDFSGLPSGFPTVSLVDSTLVEVVTASTASELEGRGTIGISGAGEISGLPSSELDISGRASGLPSGTELSGQASGSPDVSGEIPGLFGVSGQPSGFPDTSGETSGVTELSGLSSGQPGISGEASGVLYGTSQPFGITDLSGETSGVPDLSGQPSGLPGFSGATSGVPDLVSGTTSGSGESSGITFVDTSLVEVAPTTFKEEEGLGSVELSGLPSGEADLSGKSGMVDVSGQFSGTVDSSGFTSQTPEFSGLPSGIAEVSGESSRAEIGSSLPSGAYYGSGTPSSFPTVSLVDRTLVESVTQAPTAQEAGEGPSGILELSGAHSGAPDMSGEHSGFLDLSGLQSGLIEPSGEPPGTPYFSGDFASTTNVSGESSVAMGTSGEASGLPEVTLITSEFVEGVTEPTISQELGQRPPVTHTPQLFESSGKVSTAGDISGATPVLPGSGVEVSSVPESSSETSAYPEAGFGASAAPEASREDSGSPDLSETTSAFHEANLERSSGLGVSGSTLTFQEGEASAAPEVSGESTTTSDVGTEAPGLPSATPTASGDRTEISGDLSGHTSQLGVVISTSIPESEWTQQTQRPAETHLEIESSSLLYSGEETHTVETATSPTDASIPASPEWKRESESTAAAPARSCAEEPCGAGTCKETEGHVICLCPPGYTGEHCNIDQEVCEEGWNKYQGHCYRHFPDRETWVDAERRCREQQSHLSSIVTPEEQEFVNNNAQDYQWIGLNDRTIEGDFRWSDGHPMQFENWRPNQPDNFFAAGEDCVVMIWHEKGEWNDVPCNYHLPFTCKKGTVACGEPPVVEHARTFGQKKDRYEINSLVRYQCTEGFVQRHMPTIRCQPSGHWEEPQITCTDPTTYKRRLQKRSSRHPRRSRPSTAH

SEQ ID NO: 24 - protein, 680 aa, Homo sapiens
MLPQIPFLLLVSLNLVHGVFYAERYQMPTGIKGPLPNTKTQFFIPYTIKSKGIAVRGEQGTPGPPGPAGPRGHPGPSGPPGKPGYGSPGLQGEPGLPGPPGPSAVGKPGVPGLPGKPGERGPYGPKGDVGPAGLPGPRGPPGPPGIPGPAGISVPGKPGQQGPTGAPGPRGFPGEKGAPGVPGMNGQKGEMGYGAPGRPGERGLPGPQGPTGPSGPPGVGKRGENGVPGQPGIKGDRGFPGEMGPIGPPGPQGPPGERGPEGIGKPGAAGAPGQPGIPGTKGLPGAPGIAGPPGPPGFGKPGLPGLKGERGPAGLPGGPGAKGEQGPAGLPGKPGLTGPPGNMGPQGPKGIPGSHGLPGPKGETGPAGPAGYPGAKGERGSPGSDGKPGYPGKPGLDGPKGNPGLPGPKGDPGVGGPPGLPGPVGPAGAKGMPGHNGEAGPRGAPGIPGTRGPIGPPGIPGFPGSKGDPGSPGPPGPAGIATKGLNGPTGPPGPPGPRGHSGEPGLPGPPGPPGPPGQAVMPEGFIKAGQRPSLSGTPLVSANQGVTGMPVSAFTVILSKAYPAIGTPIPFDKILYNRQQHYDPRTGIFTCQIPGIYYFSYHVHVKGTHVWVGLYKNGTPVMYTYDEYTKGYLDQASGSAIIDLTENDQVWLQLPNAESNGLYSSEYVHSSFSGFLVAPM